(19) United States
(12) Patent Application Publication
(10) Pub. No.: US 2009/0099640 A1
(43) Pub. Date: Apr. 16, 2009

(54) Axial Pullwire Tension Mechanism for Self-Expanding Stent

(76) Inventor: Ning Weng, Shanghai (CN)

Correspondence Address: GLOBAL IP SERVICES, 7285 W. Eagle Court, Winton, CA, 95388, US

(21) Appl. No.: 12/295,059

(22) PCT Filed: Mar. 28, 2007
(86) PCT No.: PCT/CN07/01016

§ 371 Date: Sep. 29, 2008

(51) Int. Cl. A61F 2/06

(52) U.S. Cl. 623/1.11; 623/1.15; 623/1.12

(57) ABSTRACT

An axial pull wire tension mechanism for a self expanding stent includes a delivery system composed of an inner tube (2), a middle tube (5) and a lock wire (3), open wire knees (102) and/or close wire eyelets (103) at the both ends of the stent, and pull wires (4) for tensioning the stent. The pull wires (4) include at least one distal pull wire (42) and at least one proximal pull wire (43). A pull wire ring (421, 431) is provided at the distal end of each of the pull wires. Each pull wire passes through an opening of the inner tubing head (7) or the inner tube (2) or the middle tube (S) after the pull wire ring at its distal end is threaded through and locked temporarily by the lock wire, and travels between the open wire knees (102) or the close wire eyelets (103) at one end of the stent to constitute a temporary stent connection, thus forming the pull wire tension mechanism that can axially tension the stent. The present invention can locate the self-expanding stent in terms of its axial and rotational positions with great precision when in collaboration with the delivery system and the radially compression mechanism during the process of delivering the self-expanding stent into the patient's body, and is capable of either further adjustment should the position prove to be less ideal, or recycling should the stent prove to be incongruous after the expansion of the stent.

### TECHNICAL FIELD

The present invention generally relates to a tension mechanism employed in the delivery of different stents implanted into cardiac blood vessels and other luminal human organs; particularly refers to an axial pull wire tension mechanism for self-expanding stents.

### BACKGROUND ART

As the most important functioning human organ, the heart consists of the right chamber and the left chamber, of which each includes an atrium and a ventricle. The inter-ventricular septum separates the two ventricles whereas the inter-arterial septum divides the two atria. There is also a septum between the atrium and ventricle on each side. Four heart valves, i.e., tricuspid valve, pulmonic valve, mitral (bicuspid) valve and aortic valve, play a crucial role in the blood circulation system, wherein the anoxic blood from all body tissues enters the right atrium through superior vena cava and the inferior vena cava, and then passes through the right ventricle via tricuspid valve, where it is pumped into the pulmonary circulation system via pulmonic valve. After saturated with oxygen in lung, the blood goes back to the left atrium through pulmonary vein and enters the left ventricle via mitral valve. The subsequent ventricular systole pumps the blood through aortic valve into the aorta, where it rejoins the systemic circulation. The right and left coronary artery openings are located under the aortic valve. The unidirectional structure of the four heart valves, with the valves open when the blood is moving in a forward direction and closed when the blood is going the other way round, effectively minimizes the heart burden caused by blood counter-flow. However, due to various uncertain elements, heart valves are vulnerable to acquired injuries and are subject to pathological complications, typically rheumatism and atherosclerosis. The remote post-surgery complications of congenital heart diseases, or more specifically, Fallot tetralogy, might also contribute to pathological changes of the pulmonary valve. The clinical manifestation of the valve lesion is the gradual functional failure characterized by the blood counter-flow caused by incomplete valve shutdown, the clogged blood circulation due to narrow valves, and the heart failure conspired by the above culprits. The traditional treatment of acquired injuries or pathological changes is thoracotomy, wherein the valve lesion is surgically repaired or replaced by an artificial one while the patient is on support of hypothermic extracorporeal blood circulation after his heartbeat stops. Current artificial heart valves include metal-mechanic valves and biological valves, of which the latter is made of processed bovine pericardium, bovine jugular vein valve and porcine aortic valve. The thoracotomy as mentioned above is plagued with prolonged surgical time consumption, sky-high costs, substantial physical trauma and great risks. In addition, the metal-mechanical valve requires the patients to go through long-term anti-coagulation therapy, while extra surgeries are rife for patients using biological valves due to its limited lifespan.

In an attempt to tackle problems arising from thoracotomy in heart valve treatment, percutaneous intervention or minimally invasive surgery has been employed to implant artificial heart valves as an alternative to open-heart surgeries. Existing technologies of artificial heart valves include balloon expanding stent and self-expanding stent, of which the latter consists of a radially transformable stent that automatically expands under radial compression. To implant the artificial heart valves with self-expanding stent through blood vessels, prior protective radial compression is required until the valve is readily in place for expansion, which necessitates the design of a compression mechanism that is capable of not just radially compressing the valve prior to the implantation to facilitate the transportation but also easily decompressing the valve after it is in place for prompt expansion.

Three protocols, as mentioned below, are employed for radial compression of self-expanding stents, self-expanding stents with membrane and valves with self-expanding stents to reduce their diameters so that they can be implanted via minimally invasive surgery.

### a) Sheath Tube

Self-expanding stents, self-expanding stents with membranes and valves with self-expanding stents are radially compressed in a laterally sealed thin sheath tube made of PEFE and FEP, which, when being retracted, would trigger radial expansion of the stent. The defects of the sheath tube are that: 1. current sheath tubes for the delivery system and the radially compressed stents are hard and are of poor bending flexure; 2. friction forces between the self-expanding stent and the sheath tube in the delivery system is too big for the accurate placement of the stent and that the retraction of the sheath tube tends to encounter great resistance when the self-expanding stent is highly compressed, which results in the displacement of the stent; 3. the laterally sealed thin sheath tube blocks the guide wire that is intended to pass through the compressed stent in an inside-out or outside-in fashion; even if there were sideline openings on the sheath tube, the guide wire would have blocked the retraction of sheath tube and hence restrained the radial expansion of the stent; 4. to guarantee its mechanical strength, the minimum thickness of current sheath tubes is 0.2 to 0.3 mm, which introduces at least an extra of 0.4 to 0.6 mm in the diameter of the radially compressed stent and the delivery system.
b) External Peer-Away Membranes that can be Further Subcategorized into:
b1) peer-away membranes that are permanently fixed on the stents. A vertically placed peer-away membrane is fixed on the outer surface of the stent, wherein the membrane appears a thin tube with its opening closed and the stent inside radially compressed. When the membrane is vertically ripped open, the constraint to the stent is lifted to allow the stent to radially expand. The membrane and the stent are of identical length, which is kept constant during the compression and the expansion process, and therefore tends to clog the blood circulation that passes through the stent and flows towards lateral branch.
b2) removable external peer-away membranes
   The peer-away membrane with the radially compressed stent outperforms (a) in its bending flexure; however, defects exist in that: 1. the peer-away membrane blocks the guide wire to pass through the radially compressed stent from the sideline openings in an inside-out or outside-in fashion; the opening of the membrane serves as the only situs the guide wire can pass through, hence severely confining the rotation of the wire; 2. there have been cases in which the length of the stent is reduced after radial expansion that ensues the peering of membrane whereas the length of the membrane remain unchanged. The discrepancy in length, especially the extra length of the membrane, tends to impede the blood circulation; 3. When the peer-away membrane attached to the stent is ripped open, the stent undergoes radial expansion under the restoring forces intrinsic to the stent. As the circumference of the stent grows, the ripped membrane partially covers the outer surface of the stent on each cross-section, blocking the collateral vessels, and therefore disqualifying the stent from such applications as the aortic valve stents for coronary artery openings, thoracic aortic stents with membranes for carotid artery openings, and abdominal aortic stents with membranes for the left and right renal artery openings.
c) Stent-tying delivery system
   The method employs a soft thread that convolves the stent in all directions to radially compress the stent (ref: Chinese patent 20040054347.0)—a successful amelioration compared with the previous two methods. However, defects still exist in that: 1. the radial compression occurs only where the stent is encircled by the thread, which leads to insufficient holistic compression in the case where the stent is too long; or to complicate structure in the case where extra thread is employed to cover the whole stent. Collaboration with sheath tubes is sometimes required to offer extra radial compression, thereby tarnishing the superiority of an open structure; 2. there are chances that the thread slips off the ends of the stent in the case where it only circles the outer surface of the stent, whereas the friction forces with the stent grows in the case where the thread travels between the inner and outer surface of the stent to ward off slippage.

In addition, problems arise when the current delivery system is applied in carrying the self-expanding stents:
1. Positioning obstacles: current delivery system for intervention stent valves and the stent valves under radial compression are of rigid structure and poor bending flexure, making it hard to target the biological aortic valve opening after passing through the aortic arch. Upstream and downstream positioning and rotational positioning of the intervention stent valves and its delivery system are made difficult due to the instability of the artificial valves under the impact of the blood stream.
2. Position changes: when the self-expanding stent valve is highly compressed, the retraction of the conventional sheath tube is subject to great resistance, causing displacement of the artificial stent valve that is already in place. Upon releasing, it takes over a heartbeat cycle for the stent valve to unfold from semi-expansion to full dilations. The expanding stent valve tends to block the blood stream whose impact in turn might cause the displacement of the stent valve.
3. Position readjustment: after the full radial expansion, the position of the self-expanding stent cannot be readjusted in the case where it is mistakenly or improperly placed. The displacement may refer to upward or downward shifts in the axial direction, or rotational errors along the stent axis, or angular errors between the stent axis and the blood vessels.
4. Recycling of the stent: After the full radial expansion, the self-expanding stent cannot be retrieved should the stent prove to be incongruous.

### SUMMARY OF THE INVENTION

The present invention aims to resolve the above problems arising from the radial compression and delivery process of the current self-expanding stents by introducing an axial pull wire tension mechanism for self-expanding stents.

The present invention fulfills its objective by introducing an axial pull wire tension mechanism for self-expanding stents specializing in axially tensioning the stent when the stents is being implanted into the cardiac blood vessels. Said self-expanding stents consists of a transformable tubular net structure in which net fibers intertwine to form a plurality of transformable units, whereas both ends of the stent comprises multiple open wire knees and sealed wire eyelets; said self-expanding stents can be used in a variety of delivery system in which the stent is radially compressed, implanted in the cardiac blood vessels and then expended; said delivery system is composed of a inner tube, an optional middle tube, a proximal controller, pull wires and lock wires, the distal end of inner tubes is conjunct with inner tubing heads whereas the outer flank of its distal end is optionally conjunct with sidelining texturing tubes, with the middle tube sliding along the outer layer of the inner tube, proximal end controller is fixed at the proximal ends of both inner and middle tubes with guy ports at each end, lock wire is set in the layer between the inner tube and the middle tube.

Said axial pull wire tension mechanism is composed of inner tubes, middle tubes, lock wires, open wire knees and sealed wire eyelets at the ends of the stent that together assemble the delivery system, and pull wires for tensioning the stent; said inner tubing head shall at least have one proximal-sideline openings at the proximal end and at least one distal sideline and one proximal sideline openings at the distal end; said middle tube shall have one distal end opening and at least one optional distal sideline openings, whereas said pull wire shall at least consist of one distal end pull wire at the conjunction with the distal end of the stent and one proximal end pull wire at the conjunction with the proximal end of the stent, with a pull wire ring at the distal end of each pull wire. With pull wire ring at the distal end being threaded through and temporarily locked up by lock wires, pull wires pass through the tubing heads or the openings of the inner/middle tubes and travel between the open wire knees at the distal/proximal end of the stent, sealed wire eyelets at the ends of middle of the stent, and the transformable units in the middle to produce a temporary network that spawns the pull wire tension mechanism capable of radially tensioning the stent.

In the case where there is one said distal and proximal pull wire, respectively, of which the distal pull wire, after being locked by lock wire in the inner tube at the distal pull wire ring, passes through a sideline opening or a distal opening of the inner tube, and travels through one or two open wire knees or a sealed wire eyelet to form a temporary conjunction at the distal end of the stent, whereas the proximal pull wire, after being locked at the distal pull wire ring by lock wire in the inner tube or between the inner and middle tubes, goes through a proximal opening of the inner tube or a distal opening of the middle tube and then travels through one or two open wire knees or a sealed wire eyelet at the proximal end of the stent to form a temporary proximal conjunction. The open wire knees or sealed wire eyelets through which said distal pull wires or proximal pull wires pass can be located in a longitudinal alignment or placed with certain angles.

In the case where there are two said distal and proximal pull wires, respectively, of which the two distal pull wires, after being locked at the distal pull wire ring by the same/or different lock wires in the inner tube, thread a sideline opening or the same/or different distal openings of the inner tube, and then pass through one or two open wire knees or a sealed wire eyelet at the distal end of the stent to form two independent temporary distal conjunction of the stent, whereas the two proximal pull wire, after being locked at the distal pull wire ring by lock wires in the inner tube or between the inner and middle tubes, respectively, goes through a proximal opening of the inner tube or a distal opening of the middle tube, and then travel through one or two open wire knees or a sealed wire eyelet at the proximal end of the stent to form two independent temporary conjunctions at the proximal end of the stent. The open wire knees or the sealed wire eyelet through which the said two distal pull wires pass are located at the relative two sides of the distal end of the stent, whereas the open wire knees or the sealed wire eyelet through which the said two proximal pull wires pass are located at the relative two sides of the proximal end of the stent and are in vertical alignment or are perpendicular with the open wire knees or the sealed wire eyelet through which the two distal pull wires pass.

In the case where there are three said distal and proximal pull wires respectively, of which the three distal pull wires, after being locked respectively at the distal pull wire ring by the same/or different lock wires in the inner tube, thread a sideline opening or the same/or different distal openings of the inner tube, and then travel through one or two open wire knees or a sealed wire eyelets to form three independent temporary conjunctions at the distal end of the stent, whereas the three proximal pull wires, after being locked respectively at the distal pull wire ring by lock wires in the inner tube or between the inner and middle tubes, thread the same/or different sideline openings of the inner tube, or the same/different distal openings of the middle tube, and then travel through one or two open wire knees or a sealed wire eyelet to form three independent temporary conjunctions at the proximal end of the stent, the open wire knees or sealed wire eyelet through which the said three distal pull wires travel are equiangularly located at the distal end of the stent, whereas the open wire knees or the sealed wire eyelets through which the three said proximal pull wire pass are equiangularly located at the proximal end of the stent, and are in vertical alignment or a 60-degree angle with the open wire knees or the sealed wire eyelet through which the three distal pull wires pass.

In the case where there is one distal and proximal pull wire respectively, of which the distal pull wire, after being locked at the distal pull wire ring by the lock wire in the inner tube, threads through a sideline opening of the inner tubing head or a distal sideline opening of the inner tube, and travels through one or two open wire knees or a sealed wire eyelets at the distal end of the stent before going through another one or two open wire knees or a sealed wire eyelet at the distal end of the stent to form a secondary temporary conjunction of the distal end of the stent; whereas the proximal pull wire, after being locked at the distal pull wire ring by locked wires in the inner tube or between the inner and middle tubes, threads through a proximal opening of the inner tube or a distal opening of the middle tube, and travels through one or two open wire knees or a sealed wire eyelet at the proximal end of the stent before going through another one or two open wire knees or a sealed wire eyelet at the proximal end of the stent to form a temporary secondary conjunction at the proximal end of the stent, the open wire knees and the sealed wire eyelet through which the said distal pull wire passes in the secondary structure are located in the relative two sides of the distal end of the stent, whereas open wire knees or sealed wire eyelet through which the proximal pull wire passes in the secondary structure are located in the relative two sides at the proximal end of the stent, and are in alignment or are approximately perpendicular with the two open wire knees or sealed wire eyelets through which the distal pull wires pass in the secondary structure.

Upon completion of the temporary conjunction at the distal end of the stent, the said distal pull wire may travel through into inner tube or into the interlayer between the stent and the inner tube via the same/or different distal sideline opening of the inner tube before coming out through pull wire openings at the proximal end of the inner tube or middle tube, whereas the proximal pull wire, upon completion of the temporary conjunction at the proximal end of the stent, may travel into the inner tube through the same/or different proximal sideline openings of the inner tube, or into the interlayer between the middle tube and the inner tube through distal openings or distal sideline openings of the middle tube before coming out through pull wire opening at the proximal end of the inner tube or the middle tube.

Said two or three distal pull wires can be pulled out respectively through pull wire openings at the proximal end of the inner tube or middle tube and controlled separately to trigger the asymmetrical expansion at the distal end of the stent; whereas the said two or three proximal pull wires can be pulled out respectively through pull wire openings at the proximal end of the inner tube or middle tube and controlled separately to spark the asymmetrical expansion at the proximal end of the stent.

Said two or three distal pull wires may conglomerate to form a bus line anywhere between the temporary conjunction at the distal end of the stent and the distal end of the pull wire, through the control of which the distal end of the stent undergoes symmetrical expansion, whereas the said two or three proximal pull wires may conglomerate to form a bus line anywhere between the temporary conjunction at the proximal end of the stent and the proximal end of the pull wire, through the control of which the proximal end of the stent undergoes symmetrical expansion; by pulling and pushing the bus lines of the distal and proximal pull wires, the distal or proximal end of the stent undergoes asymmetrical expansion.

The proximal end of a said distal pull wire may merge with the proximal end of a proximal pull wire, thereby substantiating the idea to simultaneously control the distal and proximal pull wires to axially tension the stent by pulling the proximal end of just one wire.

Said bus lines of all distal pull wires and proximal pull wires may merge, thereby allowing the stent to relax or tension almost symmetrically by pulling or pushing just one bus line that controls all distal and proximal pull wires.

The above structure also include an inner tube line for distal direction control that is sliding in the lock wire cavity or outside the inner tube and whose distal end is connected with the distal end of the inner tube, with its proximal end threading through the proximal end of the inner tube and being free.

### BRIEF DESCRIPTION OF THE DRAWINGS

FIG. 1 is a three dimensional perspective view, wherein the present invention, titled the axial pull wire tension mechanism for self-expanding stent, is in joint application with the tension mechanism for flexible connecting rings;

FIG. 2 is a three dimensional perspective view, wherein the present invention, titled the axial pull wire tension mechanism for self-expanding stent, is in joint application with take-up tensioning mechanism;

FIG. 3 is a three dimensional perspective view that describes the components of the present invention, titled axial pull wire tension mechanism for self-expanding stent, and its connectors, with other radial tensioning mechanism disengaged;

FIG. 4 is a three dimensional perspective view that describes the present invention, titled axial pull wire tension mechanism for self-expanding stent, and its connectors, with the pull wires relaxed and the stent radially expanded;

FIG. 5 is a three dimensional perspective view that describes the present invention, titled axial pull wire tension mechanism for self-expanding stent, and its connectors, with lock wires that fix the pull wire disengaged;

FIGS. 6a, 6b, and 6c are sectional views that describes the present invention, titled axial pull wire tension mechanism for self-expanding stent, and its relevant connectors, wherein FIGS. 6b and 6c are variations of FIG. 6a with different ways of wire connections to the proximal pull wires at the proximal end of the stent;

FIGS. 7a, 7b and 7c are sectional views that describe a case study of the present invention, titled axial pull wire tension mechanism for self-expanding stent, and its relevant connectors, wherein FIGS. 7b and 7c are variations of FIG. 7a with different ways of wire connections to the proximal pull wire at the proximal end of the stent;

FIGS. 8a, 8b and 8c are sectional views that describe a case study of the present invention, titled axial pull wire tension mechanism for self-expanding stent, and its relevant connectors, wherein FIGS. 8b and 8c are variations of FIG. 8a with different ways of wire connections to the proximal pull wire at the proximal end of the stent;

FIG. 9 is a sectional view that describes a case study of the present invention, titled axial pull wire tension mechanism for self-expanding stent, and its relevant connectors.

### DETAIL DESCRIPTION OF THE INVENTION

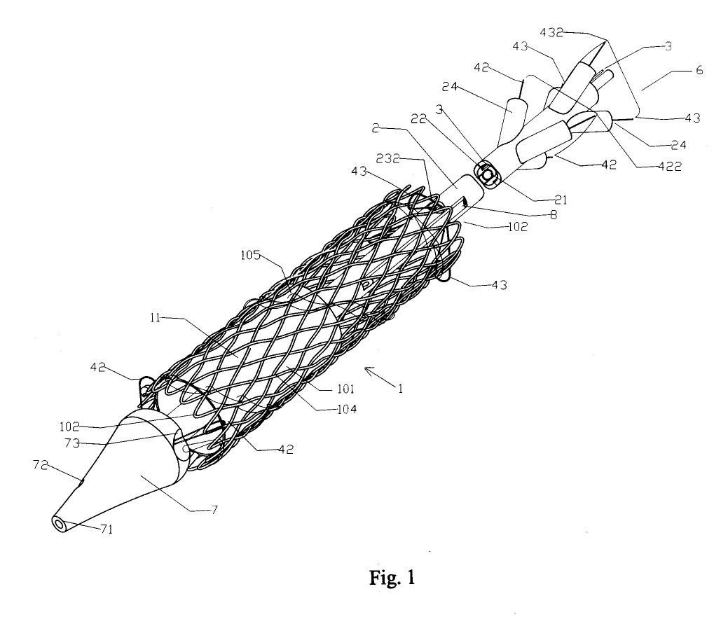

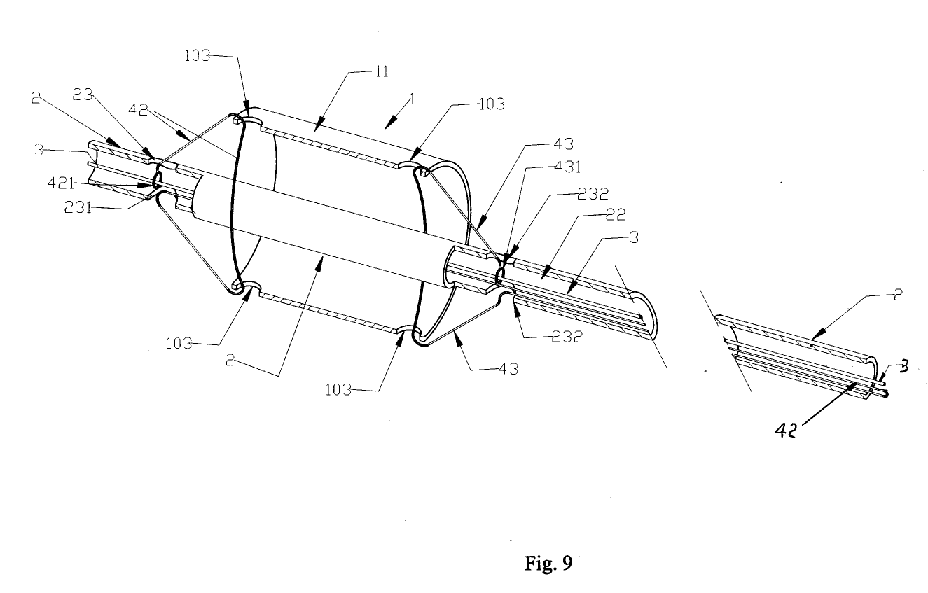

With reference to FIG. 1˜FIG. 9, the present invention, titled axial pull wire tension mechanism for self-expanding stent, is applied in the implantation of the self-expanding stent into cardiac blood vessels where it tensions the stent in collaboration with the delivery system and other stent tensioning mechanisms, of which the latter specifically refers to the flexible coupling ring tensioning mechanism and the take-up tensioning mechanism, both owned by the applicant of the present invention. (Refer to the other two patents of the applicant: “flexible coupling ring tensioning mechanism for self-expanding stent” and “take-up tensioning mechanism for self-expanding stent”)

The self-expanding stent designated as 1 in the present invention includes self-expanding stents with membranes and self-expanding stent valves, of which the former is partially or entirely covered by membranes 11 on the walls, whereas the latter, aside from being partially or entirely covered with membranes 11, is fixed with valves 12 that allow only the unidirectional passage of the blood stream (ref. FIG. 1 to FIG. 5).

The self-expanding stent features a radially deformable tubular net structure, in particular, the single-thread weaving stent composed of elastic materials like nitinol and phynox, and can be structurally subcategorized into distal and proximal ends, defined by their relative locations to the surgical patients, wherein the end in relative proximity to the patient is termed proximal, while the relatively distant end is termed distal (The rules also apply in the nomenclature of other parts in the delivery system.). The self-expanding stent undergoes radial compression under external forces and is capable of spontaneous radial expansion when the external forces are removed. The wires between the self-expanding stent weave to form a plurality of transformable units 101, and a variety of open wire knees 102 at both ends; sealed wire eyelets 103 predominate the ends and the middle area of the stent, whereas wires on the stent cross-pass each other form a plurality of intersections 104. Self-expanding stents can either be weaved by threads or cut and mould from pipes. The weaving threads are either single lines or multiple line-segments, with single lines preferred, whereas the weaving lines at the adjacent intersections are conversely connected. The stent can be single layer, sandwich layers with free tongue-shaped protrusion, or stents with branches.

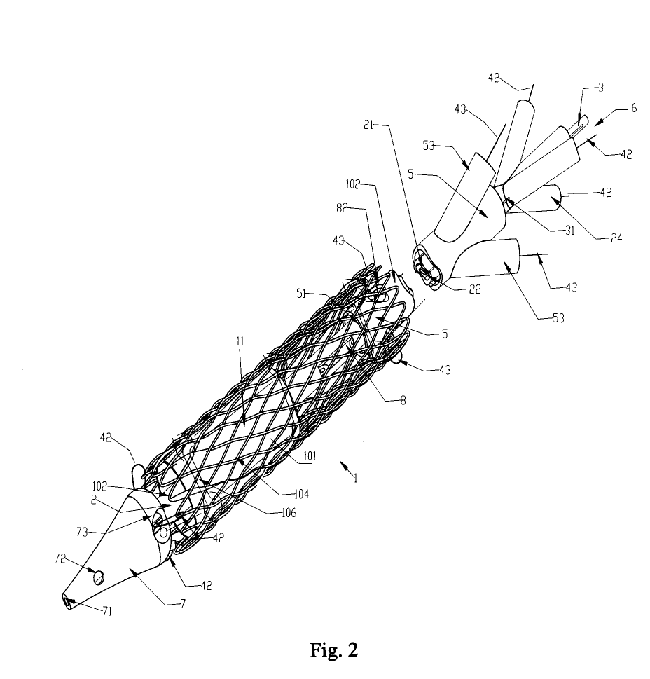

The self-expanding stent designated as 1 in the present invention can be connected to flexible coupling ring tensioning mechanism 105 (see FIG. 1) or take-up tensioning mechanism 106 (see FIG. 2), wherein the flexible coupling rings and take-up lines are weaved by such soft deformable threads as Dacron, polyethylene, PA and polypropylene.

The delivery system according to the present invention includes inner tube 2, lock wire 3, pull wire 4, middle tube 5 and proximal end controller 6.

Inner tube 2 is an acerose, bendable tube, transparent or quasi-transparent preferably, which consists of a circular 0.035″ guide wire cavity 21 and one/or multiple lock wire pulling cavities 22 that perforates from the distal end to the proximal end, wherein different types of pull wire 4 and lock wire 3 shuttle through. The distal end of inner tube 2 shall have at least one sideline openings on the cross sections from the distant to the near (distal sideline opening 231 and proximal sideline opening 232) that leads to one/or more lock wire pulling cavities 22. The sideline openings are on one or different cross sections with openings facing in one or more directions, wherein the distance between the two cross sections is larger than or equals to the length of stent 1 under radial compression. There might be one to three sideline openings on each cross section, wherein the angle between the two or more sideline openings on the same cross section is given (e.g. 120 degree). The sideline openings on the same side of the inner tube might align on the same cross section, thereby weakening the strength of the inner tube; in contrast, those sideline openings might also spatter on different cross sections that are distant apart, thereby dispersing the weak spots over the inner tube. The relative positions of the stent and the duct can be adjusted and balanced via readjusting the length of the pull wire.

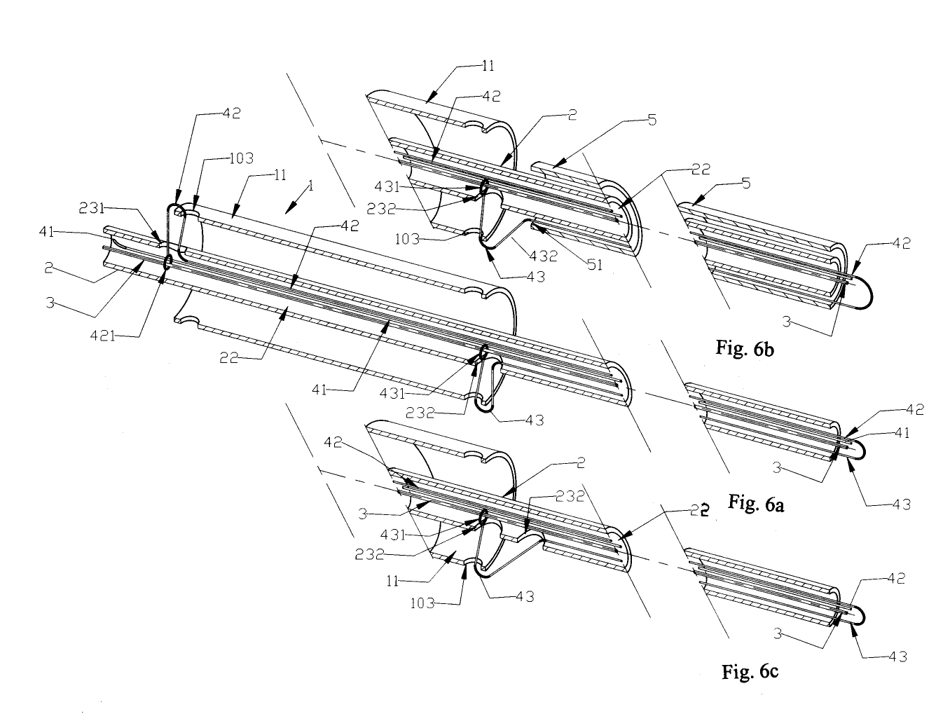

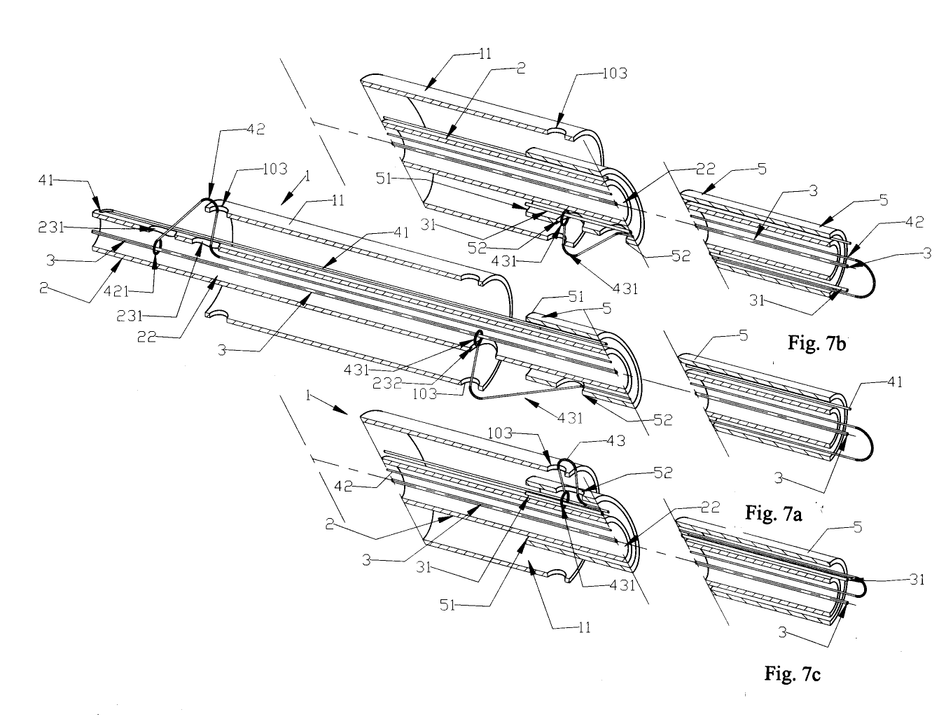

The distal end of the inner tube is attached to a cone-shaped or streamlined inner tubing head 7, on the distal end of which the guide wire opening 71 is connected to a thick guide wire cavity 21, and the rinse opening 72 in the middle is connected to the lock wire pulling cavity 22 in the inner tube. One or more sideline openings 73 are set on the same/or different cross sections at the proximal end of inner tubing head 7. In the present invention, sideline opening 73 on the inner tubing head can function as an alternative to sideline opening 231 at the distal end of the inner tube, where optional sideline guide wire duct 8 is in place. The sideline guide wire duct or guide wire may come through openings in multi-directions on the same cross section. The aperture of the guide wire duct 8 on the sideline allows guide wire at least with a diameter of less than 0.014″ to pass through. At least one lock wire 3 is placed to slide along the inner tube in the lock wire pulling cavity 22. The proximal end of the lock wire goes through the distal controller of the delivery system whereas the distal end of the inner tube may choose to install wire 41 for distal direction control (see FIGS. 6a and 7a) which may slide either inside the lock wire pulling cavity 22 or outside the inner tube. The distal end of wire 41 for distal direction control is firmly attached to the distal end of the inner tube, whereas the proximal end of the wire freely goes through the proximal end of the inner tube.

Proximal pull wire openings 24 are present at the proximal end of inner tube 2 (see FIG. 1 through FIG. 5), with their directions and quantity either identical with or different from that of the sideline openings (73, 231, 232). In the surgery, the proximal end of the inner tube stays outside the patient's body while the rest parts may proceed in vivo.

In the vicinity of proximal sideline opening 232 on the outer surface of the inner tube, a coaxial middle tube 5 can be optionally installed, whose inner diameter is larger than the outer diameter of inner tube 2 so that the middle tube can slide and rotate along the outer surface of inner tube 2. A distal opening 51 is set at the distal end of middle tube 5, whereas one to three distal sideline openings 52 are optionally installed on the same or different cross sections at the distal end of the middle tube; and the angle between two or more distal openings on the same cross section is preset (e.g. 120 degree). As middle tube 5 is able to slide and rotate, the distance and the angle between sideline openings 231, 232 of the inner tube and the sideline opening 52 of the middle tube are adjustable, and the middle part and the proximal end of middle tube 5 are at the same position with that of inner tube 2. Proximal pull wire openings 53 are present at the proximal end of the middle tube, with their direction and quantity either identical with or different from that of the sideline opening 52. In the surgery, the proximal end of the middle tube stays outside the patient's body while the rest parts proceed in vivo. The middle tube is dotted with singular or multiple pores, with different lock wires and pull wires traveling through each and every pore (of the middle tube with multiple pores).

Lock wire pulling cavity 22 of inner tube 2 is installed with one or more movable lock wires 3 that are made of such elastic materials as nitinol and phynox. Usually, the distal sideline opening 231 of the inner tube is surpassed by the distal end of lock wire 3, whose proximal end passes through the proximal end of the inner tube or proximal end controller 6. The inside of middle tube 5 or the interlayer between middle tube 5 and inner tube 2 is optionally equipped with one or more sliding lock wire 31, whose distal end shall be limited within the range of the distal end of the middle tube but shall exceed the sideline opening 52, if there is one, at the distal end of the middle tube. The proximal end of lock wire 31 comes through the proximal end of the middle tube.

Proximal end controller 6 is connected to the proximal end of the inner tube 2 and the middle tube 5.

Under certain circumstances, the outer part of middle tube 5 is optionally equipped with a coaxial sheath tube (not shown in Fig.), a thin tube whose inner diameter shall be larger than the outer diameter of middle tube 5, allowing the sheath to slide along middle tube 5. The inner diameter of the distal end of the sheath tube shall be slightly larger than the outer diameter of stent 1 under compression while the minimum length of the sheath shall be larger than that of stent 1 under compression. The distal end of the sheath tube may be engraved with an “X-ray opaque” sign.

The present invention, titled axial pull wire tension mechanism for self-expanding stent, consists of inner tube 2, middle tube 5 and lock wire 3 that together compose the delivery system; it also consists of open wire knee 102 and sealed wire eyelet 103 at both ends of the stent, and pull wire 4 for tensioning the stent, the latter of which consists at least of one distal pull wire 42 connected to the distal end of the stent and one proximal pull wire 43 connected to the proximal end of the stent. The distal end of each pull wire is equipped with a pull wire ring 421, 431, at which pull wires are temporarily passed through and locked by lock wires, and then thread the openings at the inner tubing head 7, or inner tube 2, or middle tube 5, respectively, before traveling through the open wire knees 102 at the distal/proximal end of the stent, or sealed wire eyelets 103 at the middle or both ends of the stent, or transformable units 101 in the middle section of the stent, to form a pull wire tensioning mechanism that will axially tension the stent.

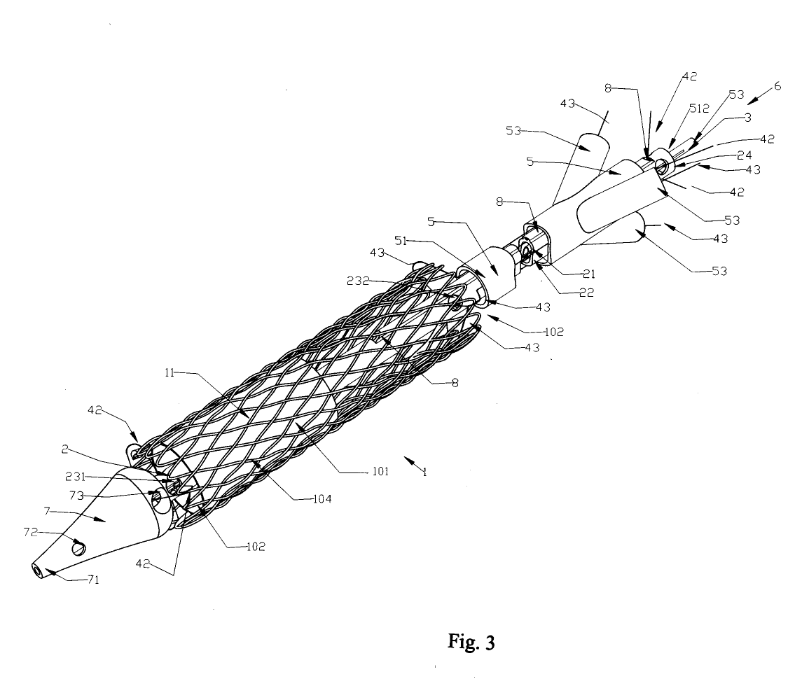

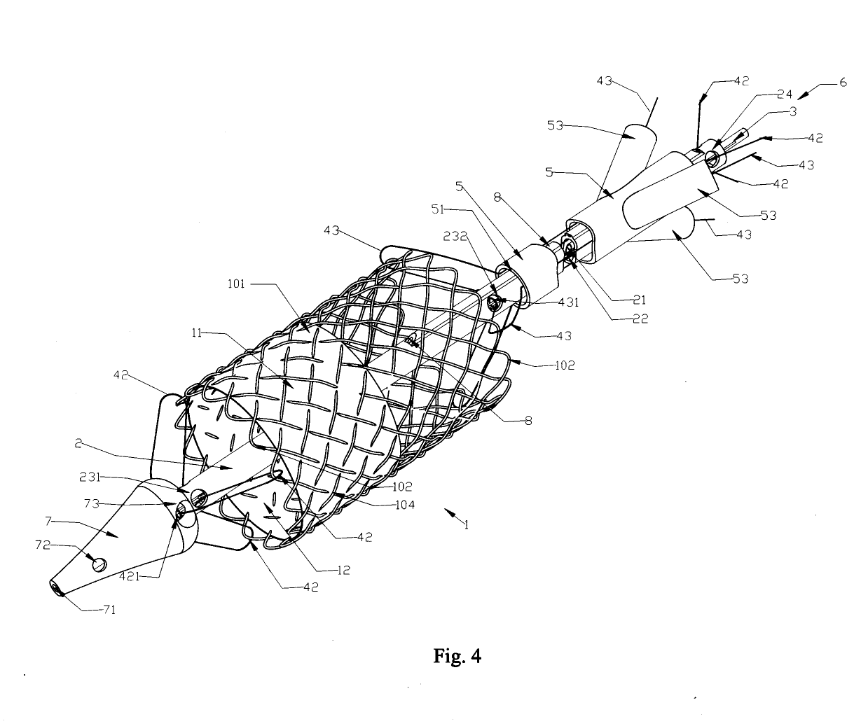

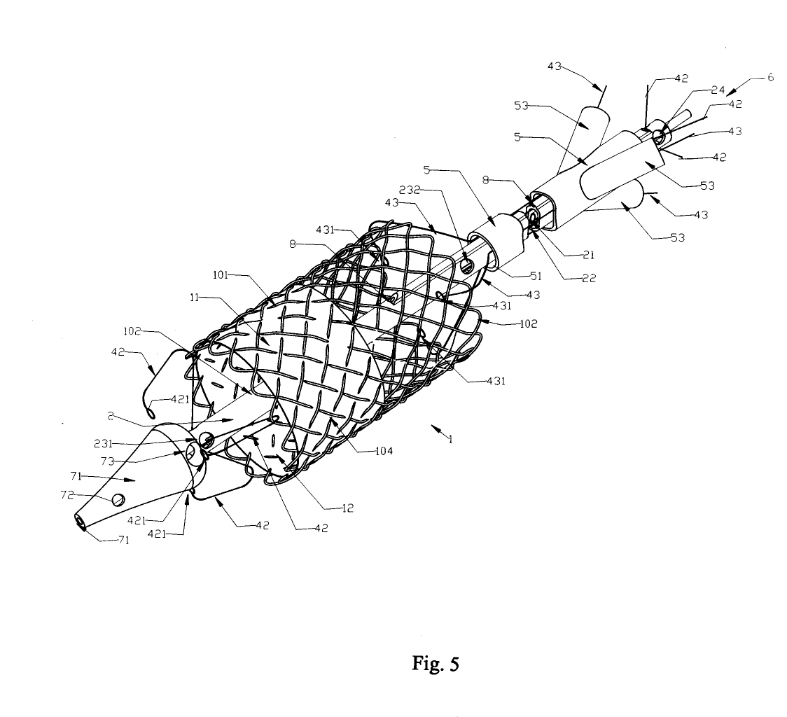

In the present invention, distal pull wire 42 and proximal pull wire 43 are usually set in pairs, wherein the distal and proximal end independently form a distal temporary conjunction and a proximal temporary conjunction to temporarily tension or loose the stent in collaboration with the delivery system when implanting the stent into the cardiac blood vessels. The work modes include:
1. In the case where there is one distal pull wire 42 and one proximal pull wire 43 (not shown), of which the distal pull wire 42, after being locked at the distal pull wire ring 421 by lock wire 3 in inner tube 2, threads a sideline opening 73 in inner tubing head 7 or a distal sideline opening 231 of inner tube 2, and passes through one or two open wire knees 102 or one sealed wire eyelet 103 at the distal end of stent 2 to form a distal temporary conjunction of the stent, whereas proximal pull wire 43, after being locked at the distal pull wire ring 431 by lock wires 3 and 31 in the inner tube 2 or between inner tube 2 and middle tube 5, threads a proximal sideline opening 232 on inner tube 2 or a distal opening 52 on the middle tube, and passes through one or two open wire knees 102 or a sealed wire eyelet 103 at the proximal end of stent 2 to form a proximal temporary conjunction of the stent, said open wire knees or sealed wire eyelet through which distal pull wire 42 and proximal pull wire 43 passes may be vertically aligned or placed with certain angles on stent 2.
2. In the case where there are two distal pull wires 42 and proximal pull wires 43, respectively (not shown), of which the two distal pull wires 42, after being locked at the distal pull wire ring 421 by the same/or different lock wire 3 in inner tube 2, thread the sideline opening 73 on the inner tubing head 7 or one or more distal opening 231 on the inner tube, and pass through one or two open wire knees 102 or a sealed wire eyelet 103 to form two independent distal temporary conjunctions for the stent, whereas the two proximal pull wires 43, after being locked at distal pull wire ring 431 by lock wire 3 and 31 respectively in inner tube 2 or between inner tube 2 and middle tube 5, thread a proximal sideline opening 232 on inner tube 2 or a distal sideline opening 52 on the middle tube, and pass through one or two open wire knees 102 or a sealed wire eyelet 103 on the proximal end of stent 2 to form two independent temporary conjunction at the proximal end of the stent, said open wire knees or sealed wire eyelets through which the two distal pull wire 42 pass are located on the relative two sides of the distal end of the stent, whereas the open wire knees or sealed wire eyelets through which the two proximal pull wires 43 pass are located on the relative two sides of the proximal end of the stent and are in longitudinal alignment with or placed with certain angles to the open wire knees or sealed wire eyelets through which the two distal pull wires pass.
3. In the case where there are three distal pull wires 42 and proximal pull wires 43, respectively, as is shown in FIG. 1, FIG. 2, FIG. 3, FIG. 4 and FIG. 5, wherein the three distal pull wires 42, after being locked at the distal pull wire ring 421 respectively by one or more lock wire 3 in inner tube 2, thread the sideline opening 73 on the inner tubing head 7 or one or more distal sideline opening 231 on the inner tube, and pass through one or two open wire knees 102 or a sealed wire eyelet 103 at the distal end of the stent to form three independent temporary conjunctions at the distal end of the stent, whereas the three proximal pull wires 43, after being locked at distal pull wire ring 431 respectively by lock wire 3 and 31 in the inner tube 2 or between inner tube 2 and middle tube 5, thread one or more proximal sideline openings on inner tube 2 or distal sideline opening 52 on the middle tube, and pass through one or two open wire knees 102 or a sealed wire eyelet 103 at the proximal end of the stent for form three independent temporary conjunctions at the proximal end of the stent, said open wire knees or sealed wire eyelet through which the three distal pull wires pass are equiangularly disposed at the distal end of the stent, whereas the open wire knees and the sealed wire eyelets through which the three proximal pull wires 43 pass are disposed equiangularly a the proximal end of the stent, and are in longitudinal alignment or placed with certain angles to the open wire knees or sealed wire eyelet through which the three distal pull wires pass.
4. In the case shown in FIG. 9 where there is one distal pull wire 42 and one proximal pull wire 43, wherein the distal pull wire 42, after being locked at the distal pull wire ring 421 by lock wire 3 in inner tube 2, threads a sideline opening 73 on inner tubing head 7 or a distal opening 231 on inner tube 2, and passes through one or two open wire knees 102 or a sealed wire eyelet 103 at the distal end of the stent, before going through another one of two open wire knees 102 or another sealed wire eyelets 103 at the distal end of stent 1 to form a secondary temporary conjunction at the distal end of the stent, whereas the proximal pull wire 43, after being locked at distal pull wire ring 431 by lock wire 3 and 31 in the inner tube 2 or between inner tube 2 and middle tube 5, thread a proximal sideline opening 232 on the inner tube or a distal sideline opening 52 on the middle tube, and passes through one or two open wire knees 102 or a sealed wire eyelet 103 at the proximal end of the stent, before going through another one or two open wire knees 102 or another sealed wire eyelet 103 at the proximal end of the stent to form a secondary temporary conjunction at the proximal end of the stent, said two open wire knees or sealed wire eyelets through which distal pull wire 42 passes for a second time are located on the relative two sides at the distal end of stent 1, whereas the two open wire knees or sealed wire eyelets through which the proximal pull wire 43 passes for a second time are located on the relative two sides at the proximal end of stent 1, and are in longitudinal alignment with or place with certain angles to the two open wire knees and sealed wire eyelets through which the distal pull wire passes for a second time.

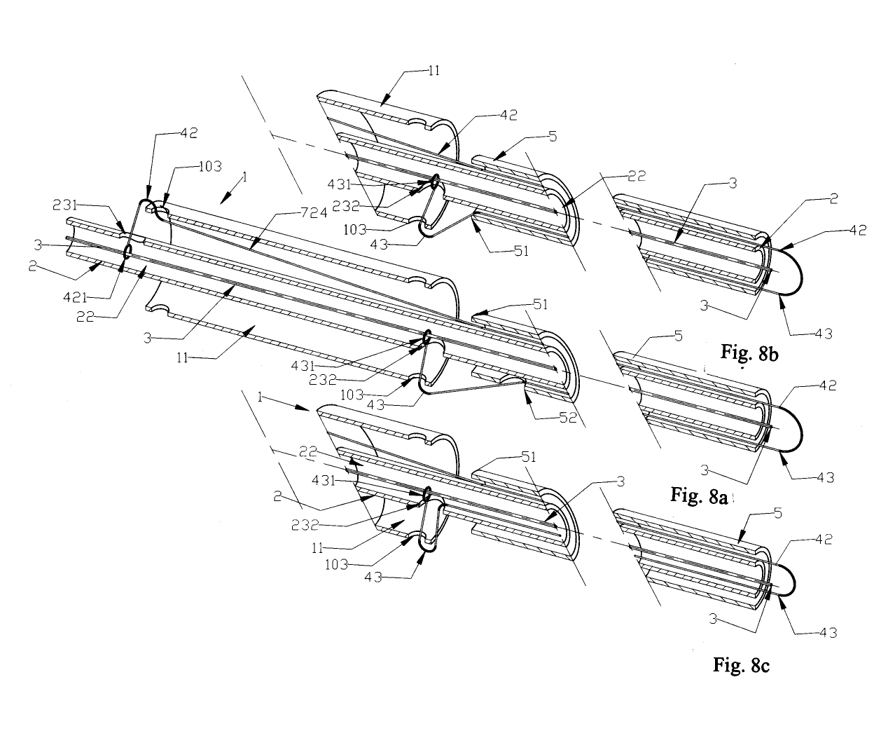

Upon completion of the temporary conjunction at the distal end of the stent, distal pull wire 42 according to the present invention may travel into inner tube 2 through the sideline opening 73 on inner tubing head 7 (shown in FIG. 1 and FIG. 2), or into inner tube 2 through the distal sideline opening 231 on the same inner tube (shown in FIG. 6a), or into inner tube 2 (shown in FIG. 7a) or interlayer between stent 1 and inner tube 2 (shown in FIG. 8a) through distal sideline opening 231 on different inner tube. The proximal end of the distal pull wire 42 can slide either in the lock wire pulling cavity 22 in the inner tube 2 (shown in FIGS. 6a and 7a), or between inner tube 2 and middle tube 5 (shown in FIG. 8a), and comes out either from the temporary pull wire opening 24 at the proximal end of the inner tube (shown in FIG. 1˜FIG. 5), or from the temporary opening 53 at the proximal end of the middle tube (not shown).

Upon completion of the temporary conjunction at the proximal end of the stent, the proximal pull wire 43 according to the present invention may travel into inner tube 2 through sideline opening 232 on the same inner tube (shown in FIG. 6a, FIG. 7c, FIG. 8c), or into inner tube 2 through sideline opening 232 on the different inner tube (shown in FIG. 6c), or into the interlayer between middle tube 5 and inner tube 2 through distal opening 51 on the middle tube (shown in FIG. 6b, FIG. 8b), or into the interlayer between middle tube 5 and inner tube 2 through distal opening 52 on the middle tube (shown in FIG. 7a, FIG. 7b and FIG. 8a). The proximal end of proximal pull wire 43 slides either in a lock wire pulling cavity 22 in inner tube 2 (shown in FIG. 6a, FIG. 6c, and FIG. 8c), or between inner tube 2 and middle tube 5 (shown in FIG. 6b, FIG. 7a, FIG. 7b, FIG. 7c, FIG. 8a, FIG. 8b) and usually comes out either from pull wire opening 53 at the proximal end of the middle tube (shown in FIG. 2˜FIG. 5), or from a temporary pull wire opening 24 at the proximal end of the inner tube (shown in FIG. 1).

In the practice of the present invention, titled axial pull wire tension mechanism for self-expanding stent, two or three distal pull wires 42 may be pulled out respectively from proximal pull wire opening 24 on the inner tube or proximal pull wire opening 53 on the middle tube and controlled accordingly, wherein the distal end of stent 1 undergoes asymmetrical expansions when tension exerted on each distal pull wire 42 is discriminated. Two or three proximal pull wires 43 may also be pulled out respectively from the proximal pull wire opening 24 on the inner tube or proximal pull wire opening 53 on the middle tube and controlled accordingly, wherein the proximal end of the stent undergoes asymmetrical expansions when tension exerted on each proximal pull wire 43 is discriminated.

In the practice of the present invention, titled axial pull wire tension mechanism for self-expanding stent, two or three distal pull wires 42 may conglomerate into a bus line 422 anywhere between the temporary conjunction at the distal end of the stent and the proximal end of the pull wire (shown in FIG. 1), wherein the proximal ends of different pull wires can be together controlled by the singular bus line at the proximal end, exerting equal tensions to each of the distal pull wires 42, thereby allowing the distal end of stent 1 to expand symmetrically. Similarly, two or three proximal pull wires 43 may conglomerate into a bus line 432 anywhere between the temporary conjunction at the proximal end of the stent and the proximal end of the pull wire (shown in FIG. 1), wherein the proximal end of different proximal pull wires can be controlled together by the singular bus line at the proximal end. By tensioning or loosening the distal pull wire bus line 422 and the proximal pull wire bus line 432, the distal and proximal ends of stent expand asymmetrically.

In the practice of the present invention, titled axial pull wire tension mechanism for self-expanding stent, the proximal end of distal pull wire 42 and that of proximal pull wire 43 may merge (not shown) so that the control of the distal end of distal pull wire 42 and that of proximal pull wire 43 can be synchronized. By axially pulling stent 1 in the opposite direction to lengthen the stent and dwindling the diameter, the stent may be axially compressed or expanded either symmetrically or asymmetrically.

In the practice of the present invention, titled axial pull wire tension mechanism for self-expanding stent, the bus line 421 of all distal pull wires 42 and that 431 of all proximal pull wires 43 may conglomerate to form a bus line (not shown) by which all distal and proximal pull wires can be controlled, thereby allowing the ends of the stent to tension or loose almost symmetrically.

In the practice of the present invention, titled axial pull wire tension mechanism for self-expanding stent, one of the two protocols below is employed to axially tension the stent:
1. Through the relative slide of pull wire against the inner or middle tube;
2. Through the relative displacement between inner tube and middle tube, whereas the relative position of each pull wire against the inner tube or middle tube remains unchanged.

The functions and the relevant working principles of the present invention, titled axial pull wire tensioning mechanism for self-expanding stent, are interpreted as follows:

Assembly: When distal pull wire 42, proximal pull wire 43, stent 1, inner tube 2, lock wire 3 and middle tube 5 are all in place, pull all the pull wires 42, 43 at the proximal end of the delivery system to length the stent 1 and dwindle its diameter.
Or, push the sheath tube towards the distal end to encompass the outer surface of the dwindling stent 1 with minimized diameter;
Or, strain and lock the flexible coupling ring tensioning mechanism 106 (shown in FIG. 1);
Or, strain and lock the take-up tensioning mechanism 106 (shown in FIG. 2)

Entry: Wash the delivery system to weed out the bubbles inside, and inject the radially compressed stent 1 and the delivery system into the target blood vessels along one or two guide wires.

Radial compression withdrawal mechanism:
Push the inner tube 2 and middle tube 5 forward towards the distal end, or pull the sheath tube backward towards the proximal end to separate the stent 1 from the sheath tube;
Or, pull lock wire 3 and 31 backward towards the proximal end to automatically disengage flexible coupling ring tensioning mechanism 105 (shown in FIG. 1)
Or, backwardly withdraw lock wire 3 and 31, pull the stent and wind the wire to sequentially disengage the take-up tensioning mechanism 106 (shown in FIG. 2)
Upon lifting the compression, the diameter of stent 1 may slightly expand, but its ends are still under the pulling traction by distal/proximal temporary pull wires 42, 43. (shown in FIG. 3)

Expansion of stent 1: When stent 1 is in place, slightly relax distal pull wire 42 and proximal pull wire 43 that are previously tensioned at the proximal end of the delivery system (i.e., the proximal end of inner tube or middle tube). Relative to inner tube 2 or middle tube 5, the distal end of distal pull wire 42 and that of proximal pull wire 43, both located outside the inner tube 2, are lengthened as the above two pull wires move towards the distal end of the inner tube under the restoring force of stent 1, whereas the length of stent 1 dwindles and its diameter expands till the stent is fully dilated, accompanying the diminishing of the longitudinal traction at both ends of stent 1. And stent 1 adhibits to vascular walls thereafter to maintain a mechanical balance. (Shown in FIG. 4, FIG. 5)

Recompression: Should stent 1 prove to be erroneously placed after the expansion, gently pull the proximal end of distal pull wire 42 and that of proximal pull wire 43 that are already in relaxation. Relative to inner tube 2 and middle tube 5, the distal end of distal pull wire 42 and that of proximal pull wire 43, both outside inner tube 2, are shortened as the above two pull wires move towards the proximal end of stent 2, whereas stent 1 is lengthened, its diameter dwindled, to a total compression, as the longitudinal traction on both ends of stent 1 grows. Stent 1 then adhibits to inner tube 2 to seek a new mechanical balance. As the diameter of stent 1 reduces to one that is smaller than that of the vascular wall, stent 1 may be moved to a new spot for further expansion.

Fine tune of expansion: In the process of tensioning and loosening pull wires 42 and 43, tensions exerted on them may vary, leading to varied degree of expansion at the distal and proximal end of stent 1, and therefore varied diameters; whereas tensions exerted on each of every distal pull wire 42 or proximal pull wire 43 may also vary, producing a set of pull wires with varying lengths. The above settings make it possible for the distal or proximal end of stent 1 to expand asymmetrically. It is not a necessity for the long axis of stent to be in parallel with that of inner tube 2.

Final release: When the size, shape and position of the dilating stent 1 are all confirmed, pull the corresponding lock wire 3 or 31 to the proximal end at the proximal end of lock wire 3 or 31, and retreat the distal end of lock wire to the proximal end of the corresponding sideline openings (73, 231, 232, 52) to disengage the locking mechanism that controls the pull wire rings or semi-rings (421, 431). Pull the proximal ends of the distal pull wire 42 and proximal pull wire 43 towards the proximal end, and retreat pull wire ring or semi-ring (421, 431) from the open wire knees 102 or sealed wire eyelets 103 at the end of the stent from which they once have previously passed through. Now that the stent 1 is completely released and totally independent of the delivery system, the position of stent 1 can no longer be readjusted and the delivery system can be extracted from the patient's body.

Recycling: When stent 1 is fully dilated but not yet finally released, distal pull wire 42 and proximal pull wire 43 are still connected to stent 1. Should stent 1 prove to be incongruous, strain all the proximal ends of distal/proximal pull wires to maximize the length of stent 1 and minimize its diameter, and push the sheath tube forward towards the distal end or push inner tube 2 and middle tube 5 backward towards the proximal end to partially contain the radially compressed stent 1 into the sheath tube.

### INDUSTRIAL APPLICATIONS

The present invention, titled axial pull wire tensioning mechanism for self-expanding stent, has the following advantages and positive effects:
1. The distal/proximal pull wire of the present invention, titled axial pull wire tensioning mechanism for self-expanding stent, only needs to pass through the distal and proximal end of the stent once, respectively, and the stent does not have to be bound with the pull wire externally. For distal/proximal pull wires that are partially outside the inner tube, the outer parts are located on an axial plane almost in parallel with or overlapped with the vertical axis of the stent or that of the inner tube. The stent is axially lengthened when the distal or the proximal pull wire at both ends of the stent are longitudinally pulled in the opposite direction. For single thread weaving stent, the axial lengthening is accompanied with concurring radial compression. In addition, for pull wires partially outside the inner tube, the inner and outer parts shall be aligned on the same plane.
2. Tensions exerted on the end of the stent by each and every distal/proximal pull wire may be identical or discriminated, whereas the layout, expansion of and the readjustment to the end of the stent may be symmetrical or asymmetrical relative to the inner and middle tube.
3. When the stent under radial compression is in place, the axial angle of the stent can be fine-tuned with the assistance of distal/proximal pull wires and/or pull wire for direction control at the distal end of the inner tube.
4. The expansion process is expeditious by just loosening the tensioned distal/proximal pull wires or pushing the middle tube forward towards the distal end.
5. When the stent is fully dilated but not yet finally released, it can still be recompressed and re-expanded, and its position is also adjustable.
6. Should the dilated stent prove incongruous, it can be recycled before the final release.
7. Friction forces are minimized with every distal/proximal pull wire aligned on the same plane

What is claimed is:

1. An axial pull wire tension mechanism for self-expanding stent employed for radially tensioning the stent during an implantation of a self-expanding stent into the cardiac blood vessels, said self-expanding stent comprises a radially transformable tubular net structure, of which net fibers intertwine to form multiple transformable units, two ends of the stent comprises a plurality of open wire knees and sealed wire eyelets, said self-expanding stent can be implanted into the cardiac blood vessels after being radially compressed by a class of open-ended delivery system that comprises inner tubes, optional middle tubes, a proximal end controller, stayguys and lock wires, a further end of the inner tubes is conjunct with an inner tubing heads whereas an outer flank of its distal end is conjunct with sidelining texturing tubes, with the middle tube sliding along the outer layer of the inner tube, the proximal end controller is fixed at proximal ends of both the inner and middle tubes with guy ports at each end, the lock wire is set in the layer between the inner tube and the middle tube;
wherein said axial pull wire tension mechanism comprises the inner tubes, the middle tubes, the lock wires, the open wire knees and the sealed wire eyelets at the ends of the stent that together assemble a delivery system, and pull wires for tensioning the stent, said inner tubes at least have one proximal-sideline openings at the proximal end and at least one distal sideline and one proximal sideline openings at the distal end, said middle tube have one distal end opening and at least one optional distal sideline opening, said pull wire at least has one distal end pull wire at the conjunction with the distal end of the stent and one proximal end pull wire at the conjunction with the proximal end of the stent, with a pull wire ring at the distal end of each pull wire, with a pull wire ring at the distal end being threaded through and temporarily locked up by lock wires, pull wires pass through the tubing heads or the openings of the inner/middle tubes and travel between the open wire knees at the distal/proximal end of the stent, sealed wire eyelets at the ends of middle of the stent, and the transformable units in the middle to produce a temporary network that spawns the pull wire tension mechanism capable of radially tensioning the stent.

2. The axial pull wire tensioning mechanism for self-expanding stent according to claim 1, wherein there is one said distal and proximal pull wire, respectively, of which the distal pull wire, after being locked by the lock wire in the inner tube at the distal pull wire ring, passes through a sideline opening of the inner tubing head or a distal opening of the inner tube, and travels through one or two open wire knees or a sealed wire eyelet to form a temporary conjunction at the distal end of the stent, whereas the proximal pull wire, after being locked at the distal pull wire ring by lock wire in the inner tube or between the inner and middle tubes, goes through a proximal opening of the inner tube or a distal opening of the middle tube and then travels through one or two open wire knees or a sealed wire eyelet at the proximal end of the stent to form a temporary conjunction at the proximal end of the stent; said open wire knees or sealed wire eyelets through which the distal pull wires or proximal pull wires pass can be located in a longitudinal alignment or placed with certain angles.

3. The axial pull wire tensioning mechanism for self-expanding stent according to claim 1, wherein there are two said distal and proximal pull wires, respectively, of which the two distal pull wires, after being locked at the distal pull wire ring by the same/or different lock wires in the inner tube, thread a sideline opening or the same/or different distal openings of the inner tube, and then pass through one or two open wire knees or a sealed wire eyelet at the distal end of the stent to form two independent temporary conjunctions at the distal end of the stent, whereas the two proximal pull wires, after being locked at the distal pull wire ring by lock wires in the inner tube or between the inner and middle tubes, respectively, go through a proximal opening of the inner tube or a distal opening of the middle tube, and then travel through one or two open wire knees or a sealed wire eyelet at the proximal end of the stent to form two independent temporary conjunctions at the proximal end of the stent; the open wire knees or the sealed wire eyelet through which said two distal pull wires pass are located at the relative two sides of the distal end of the stent, whereas the open wire knees or the sealed wire eyelet through which the said two proximal pull wires pass are located at the relative two sides of the proximal end of the stent and are in vertical alignment or are perpendicular with the open wire knees or the sealed wire eyelet through which the two distal pull wires pass.

4. The axial pull wire tension mechanism for self-expanding stent according to claim 1, wherein there are three said distal and proximal pull wires respectively, of which the three distal pull wires, after being locked respectively at the distal pull wire ring by the same/or different lock wires in the inner tube, thread a sideline opening or the same/or different distal openings of the inner tube, and then travel through one or two open wire knees or a sealed wire eyelets to form three independent temporary conjunctions at the distal end of the stent, whereas the three proximal pull wires, after being locked respectively at the distal pull wire ring by lock wires in the inner tube or between the inner and middle tubes, thread the same/or different sideline openings of the inner tube, or the same/different distal openings of the middle tube, and then travel through one or two open wire knees or a sealed wire eyelet to form three independent temporary conjunctions at the proximal end of the stent, the open wire knees or sealed wire eyelet through which the said three distal pull wires travel through are equiangularly located at the distal end of the stent, whereas the open wire knees or the sealed wire eyelets through which the three said proximal pull wire pass are equiangularly located at the proximal end of the stent, and are in vertical alignment or a 60-degree angle with the open wire knees or the sealed wire eyelet through which the three distal pull wires pass.

5. The axial pull wire tension mechanism for self-expanding stent according to claim 1, wherein there is one distal and proximal pull wire respectively, of which the distal pull wire, after being locked at the distal pull wire ring by the lock wire in the inner tube, threads through a sideline opening of the inner tubing head or a distal sideline opening of the inner tube, and travels through one or two open wire knees or a sealed wire eyelets at the distal end of the stent before going through another one or two open wire knees or a sealed wire eyelet at the distal end of the stent to form a secondary temporary conjunction of the distal end of the stent; whereas the proximal pull wire, after being locked at the distal pull wire ring by lock wires in the inner tube or between the inner and middle tubes, threads through a proximal opening of the inner tube or a distal opening of the middle tube, and travels through one or two open wire knees or a sealed wire eyelet at the proximal end of the stent before going through another one or two open wire knees or a sealed wire eyelet at the proximal end of the stent to form a temporary secondary conjunction at the proximal end of the stent, the open wire knees and the sealed wire eyelet through which the said distal pull wire passes in the secondary structure are located in the relative two sides of the distal end of the stent, whereas open wire knees or sealed wire eyelet through which the proximal pull wire passes in the secondary structure are located in the relative two sides at the proximal end of the stent, and are in alignment or are approximately perpendicular with the two open wire knees or sealed wire eyelets through which the distal pull wires pass in the secondary structure.

6. The axial pull wire tension mechanism for self-expanding stent according to claim 2 or 3 or 4 or 5, wherein said distal pull wire, upon completion of the temporary conjunction at the distal end of the stent, may travel through into inner tube or into the interlayer between the stent and the inner tube via the same/or different distal sideline opening of the inner tube before coming out through pull wire openings at the proximal end of the inner tube or middle tube, whereas the proximal pull wire, upon completion of the temporary conjunction at the proximal end of the stent, may travel into the inner tube through the same/or different proximal sideline openings of the inner tube, or into the interlayer between the middle tube and the inner tube through distal openings or distal sideline openings of the middle tube before coming out through pull wire opening at the proximal end of the inner tube or the middle tube.

7. The axial pull wire tensioning mechanism for self-expanding stent according to claim 1 or 3 or 4, wherein said two or three distal pull wires can be pulled out respectively through pull wire openings at the proximal end of the inner tube or middle tube and controlled separately to trigger the asymmetrical expansion at the distal end of the stent; whereas the said two or three proximal pull wires can be pulled out respectively through pull wire openings at the proximal end of the inner tube or middle tube and controlled separately to spark the asymmetrical expansion at the proximal end of the stent.

8. The axial pull wire tension mechanism for self-expanding stent according to claim 1 or 3 or 4, wherein said two or three distal pull wires may conglomerate to form a bus line anywhere between the temporary conjunction at the distal end of the stent and the distal end of the pull wire, through the control of which the distal end of the stent undergoes symmetrical expansion, whereas the said two or three proximal pull wires may conglomerate to form a bus line anywhere between the temporary conjunction at the proximal end of the stent and the proximal end of the pull wire, through the control of which the proximal end of the stent undergoes symmetrical expansion; by pulling and pushing the bus lines of the distal and proximal pull wires, the distal or proximal end of the stent undergoes asymmetrical expansion.

9. The axial pull wire tension mechanism for self-expanding stent according to claim 1, wherein said proximal end of a distal pull wire may merge with the proximal end of a proximal pull wire, thereby substantiating the idea to simultaneously control the distal and proximal pull wires to axially tension the stent towards both ends by pulling the proximal end of just one wire.

10. The axial pull wire tension mechanism for self-expanding stent according to claim 8, wherein said bus lines of all distal pull wires and proximal pull wires may merge, thereby allowing the stent to relax or tension almost symmetrically by pulling or pushing just one bus line that controls all distal and proximal pull wires.

11. The axial pull wire tension mechanism for self-expanding stent according to claim 1, wherein said structure above also includes an inner tube line for distal direction control that is sliding in the lock wire cavity or outside the inner tube and whose distal end is connected with the distal end of the inner tube, with its proximal end threading through the proximal end of the inner tube and being free.
